SEQUENCE LISTING (10 sequences)

SEQ ID NO: 1 - DNA, 1961 bp, Pichia guilliermondii
  CDS (502)..(1887)
accaggcatggcgagaagggtagagcatcaatgaaacgttgcactacttcaccactcgtaaattgaccagagccgcaccgacatggatgtcgattggcccgatgagcgagcattggttagcgaaattaagcccaacgatgtgatgggatctttcacggtcaacaagctcaaagtggtgttacccacggaaaagggtaaggaaaccaaaggcaaaggtgtcaaaaaaaaggggaagaaataattagacgtatagagtgcattggtgtcccaataggtacctaattaagacccgatgtagtggtgtgtgcggcgcatggtgcattgtgtcaatgcacgttgtatactccttttcataacccacatgggtagcactatataaagcgaccccaacgctactaattgattccaaactcaattgatcctgactgaattgaattgaaaaattcaagtcgcattctgaattctttcgaataattgagtcgcattttcatttcaattgtaatggtcgctattcaaaaagctctcgttcccggtctctacctcgcatccaattatcgtgatgttgctacccccgagctagccgccagagaccagtacaacattgtcaagtaccttggaggcgctgggccttatattcagtttgagggctttggaattgacacaaatgtacctgaacaatgtacggtggaactggttcaattgtacatgagacacggcgagaggtttcctgggttgtctgctggtcaacagcaacatgctttggtcaaaaaacttcaaaattacaacaaaaccattaccggtccattatcgtttttgaatgactacacctactatgttcaaaacgaagaaaactacgaattggaaaccacaccctggaacaccaattctccttacactggctacgataccgcagttaaggcgggctctgctttcagagcaaagtacaaccacttgtacaacgaaaacaaaactttaccagttttcgcagccgcttctaaaagagtatacgacactggaaacttctttgttcaaggattccttggtcccgattatctggacgagtcggttgaccatgtagttctctcggaggaagattttcttggaattaatactcttgtacctcggtggggatgtaaggctttcaattcttcgtccaacgatgaacttatagctcaatttcctagcaattatacccaagatatcgtcaagcggttgacagatggtaacgacggtttgaatcttactactaaggatgtttctaacctctttcaactttgtgcctatgagctcagtgctactggatactctccattctgcgacatctttactcaagacgaattggttttgcacagttatgccagtgatcttcagtattactacacctctggacctggaggaaacctcaccagaaccgtaggggccattcaattgaacgcttctttggctttgttgaaacaaactgaatcagacaacaaaatctggttgagttttactcacgatactgatattgaaattttccatgctgctcttggtttgtttgatccacttgaacctttacccgtcaacgaaacccgcttcagagacatgtaccaccatgtgaatgtggttcccatgggttccagaaccattactgaaaagttaaagtgtggagacgaaacctacgtaagattcattatcaacgatgcagttgttcctgttccaaagtgtcaggatggaccaggcttctcctgtaagctttcagattttgagaactatgttgctgagcgtttaagtggtatcgacattgtgaaagattgtaaggttcctgacgatgttcctcaagaattgacgttctactgggactaccagtctggacagtacaatgctactgccgaaagaattgtgcgataggggaaagtgaaagatggaaagagtgaaaaattattgagggttggaaatctttttcatgatagaagattaat

SEQ ID NO: 2 - protein, 462 aa, Pichia guilliermondii
MVAIQKALVPGLYLASNYRDVATPELAARDQYNIVKYLGGAGPYIQFEGFGIDTNVPEQCTVELVQLYMRHGERFPGLSAGQQQHALVKKLQNYNKTITGPLSFLNDYTYYVQNEENYELETTPWNTNSPYTGYDTAVKAGSAFRAKYNHLYNENKTLPVFAAASKRVYDTGNFFVQGFLGPDYLDESVDHVVLSEEDFLGINTLVPRWGCKAFNSSSNDELIAQFPSNYTQDIVKRLTDGNDGLNLTTKDVSNLFQLCAYELSATGYSPFCDIFTQDELVLHSYASDLQYYYTSGPGGNLTRTVGAIQLNASLALLKQTESDNKIWLSFTHDTDIEIFHAALGLFDPLEPLPVNETRFRDMYHHVNVVPMGSRTITEKLKCGDETYVRFIINDAVVPVPKCQDGPGFSCKLSDFENYVAERLSGIDIVKDCKVPDDVPQELTFYWDYQSGQYNATAERIVR

SEQ ID NO: 3 - DNA, 1389 bp, Pichia guilliermondii
atggtcgctattcaaaaagctctcgttcccggtctctacctcgcatccaattatcgtgatgttgctacccccgagctagccgccagagaccagtacaacattgtcaagtaccttggaggcgctgggccttatattcagtttgagggctttggaattgacacaaatgtacctgaacaatgtacggtggaactggttcaattgtacatgagacacggcgagaggtttcctgggttgtctgctggtcaacagcaacatgctttggtcaaaaaacttcaaaattacaacaaaaccattaccggtccattatcgtttttgaatgactacacctactatgttcaaaacgaagaaaactacgaattggaaaccacaccctggaacaccaattctccttacactggctacgataccgcagttaaggcgggctctgctttcagagcaaagtacaaccacttgtacaacgaaaacaaaactttaccagttttcgcagccgcttctaaaagagtatacgacactggaaacttctttgttcaaggattccttggtcccgattatctggacgagtcggttgaccatgtagttctctcggaggaagattttcttggaattaatactcttgtacctcggtggggatgtaaggctttcaattcttcgtccaacgatgaacttatagctcaatttcctagcaattatacccaagatatcgtcaagcggttgacagatggtaacgacggtttgaatcttactactaaggatgtttctaacctctttcaactttgtgcctatgagctcagtgctactggatactctccattctgcgacatctttactcaagacgaattggttttgcacagttatgccagtgatcttcagtattactacacctctggacctggaggaaacctcaccagaaccgtaggggccattcaattgaacgcttctttggctttgttgaaacaaactgaatcagacaacaaaatctggttgagttttactcacgatactgatattgaaattttccatgctgctcttggtttgtttgatccacttgaacctttacccgtcaacgaaacccgcttcagagacatgtaccaccatgtgaatgtggttcccatgggttccagaaccattactgaaaagttaaagtgtggagacgaaacctacgtaagattcattatcaacgatgcagttgttcctgttccaaagtgtcaggatggaccaggcttctcctgtaagctttcagattttgagaactatgttgctgagcgtttaagtggtatcgacattgtgaaagattgtaaggttcctgacgatgttcctcaagaattgacgttctactgggactaccagtctggacagtacaatgctactgccgaaagaattgtgcgatag

SEQ ID NO: 4 - protein, 34 aa, Pichia guilliermondii
VAIQKALVPGLYLASNYRDVATPELAARDQYNIV

SEQ ID NO: 5 - DNA, 17 bp, Pichia guilliermondii
  misc_feature (3)..(3): n is a, c, g, or t
gtngcnathcaraargc

SEQ ID NO: 6 - protein, 6 aa, Pichia guilliermondii
VAIQKA

SEQ ID NO: 7 - protein, 6 aa, Pichia guilliermondii
QNEENY

SEQ ID NO: 8 - DNA, 18 bp, Pichia guilliermondii
  misc_feature (1)..(1): r is a or g
rtarttytcytcrttytg

SEQ ID NO: 9 - DNA, 19 bp, Pichia guilliermondii
gtggttgtactttgctctg

SEQ ID NO: 10 - DNA, 18 bp, Pichia guilliermondii
cccgattatctggacgag

(19) United States
(12) Patent Application Publication
(10) Pub. No.: US 2009/0081331 A1
(43) Pub. Date: Mar. 26, 2009

(54) Phytase

(75) Inventors: Stephan Haefner, Speyer (DE); Oskar Zelder, Speyer (DE); Anja Knietsch, Ilvesheim (DE); Edzard Scholten, Mannheim (DE); Thomas Friedrich, Darmstadt (DE); Thomas Brugger, Rodersheim-Gronau (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/918,578

(22) PCT Filed: Apr. 19, 2006
(86) PCT No.: PCT/EP2006/003584

§ 371 Date: Oct. 16, 2007

(30) Foreign Application Priority Data
Apr. 21, 2005 (EP) 05008807.9

(51) Int. Cl. C12N 15/63; C07H 21/00; C12N 1/21; C12N 1/19; C12N 1/15; C12N 5/04; C12N 5/10; C12P 21/00; C12N 9/14; C07K 16/00; A23L 1/28

(52) U.S. Cl. 426/60; 536/23.2; 435/320.1; 435/440; 435/252.3; 435/254.2; 435/254.11; 435/419; 435/325; 435/69.1; 435/195; 435/254.23; 530/387.9; 435/262; 426/62

(57) ABSTRACT

Described are DNA sequences encoding a polypeptide exhibiting phytase activity, the corresponding encoded phytase polypeptide, a process for preparing the polypeptide and the use thereof for various industrial applications.

The present invention relates to DNA sequences encoding a polypeptide exhibiting phytase activity, the corresponding encoded phytase polypeptide, a process for preparing the polypeptide, and the use thereof for various industrial applications, in particular in animal feed.

Phytic acid or myo-inositol 1,2,3,4,5,6-hexakis dihydrogen phosphate (also referred to as myo-inositol hexakisphosphate) is the primary source of inositol and the primary storage form of phosphate in plant seeds. In fact, it is naturally formed during the maturation of seeds and cereal grains. In the seeds of legumes it accounts for about 70% of the phosphate content and is structurally integrated with the protein bodies as phytin, a mixed potassium, magnesium and calcium salt of inositol. Seeds, cereal grains and legumes are important components of food and feed preparations, in particular of animal feed preparations. But also in human food cereals and legumes are becoming increasingly important.

The phosphate moieties of phytic acid chelates divalent and trivalent cations such as metal ions; i.a. the nutritionally essential ions of calcium, iron, zinc and magnesium as well as the trace minerals mangane, copper and molybdenum.

Apart from that the phytic acid also to a certain extent binds proteins by electrostatic interaction. At a pH below the isoelectric point (pl) of the protein, the positively charged protein binds directly to phytate. At a pH above the pl, the negatively charged protein binds via metal ions to phytate.

Phytic acid and its salts, phytates, are often not metabolized since they are not absorbable from the gastrointestinal system, i.e. neither the phosphorous thereof, nor the chelated metal ions, nor the bound proteins are nutritionally available.

Accordingly, since phosphorus is an essential element for the growth of all organisms, food and feed preparations need to be supplemented with inorganic phosphate. Quite often also the nutritionally essential ions such as iron and calcium, must be supplemented. Moreover, the nutritional value of a given diet decreases because of the binding of proteins by phytic acid. Accordingly, phytic acid is often termed an anti-nutritional factor.

Finally, since phytic acid is not metabolized, the phytate phosphorus passes through the gastrointestinal tract of such animals and is excreted with the manure, leading to an undesirable phosphate pollution of the environment resulting, e.g., in eutrophication of the water environment and extensive growth of algae.

Phytic acid or phytates (said terms being, unless otherwise indicated, in the present context used synonymously or at random) are degradable by phytases.

In most of those plant seeds which contain phytic acid, endogenous phytase enzymes are also found. These enzymes are formed during the germination of the seed and serve the purpose of liberating phosphate and, as the final product, free myo-inositol for use during the plant growth.

When ingested, the phytates contained in food or feed components are in theory hydrolysable by the endogenous plant phytases of the seed in question, by phytases stemming from the microbial flora in the gut and by intestinal mucosal phytases. In practice, however, the hydrolyzing capability of the endogenous plant phytases and the intestinal mucosal phytases, if existing, is far from sufficient for increasing significantly the bioavailability of the bound or constituent components of phytates. However, when the process of preparing the food or feed involves germination, fermentation or soaking, the endogenous phytase might contribute to a greater extent to the degradation of phytate.

In ruminant or polygastric animals such as horses and cows the gastro intestinal system hosts microorganisms capable of degrading phytic acid. However, this is not so in monogastric animals such as human beings, poultry and swine. Therefore, the problems indicated above are primarily of importance as regards such monogastric animals.

The production of phytases by plants as well as by microorganisms has been reported. Amongst the microorganisms, phytase producing bacteria as well as phytase producing fungi are known.

From the plant kingdom, e.g. a wheat-bran phytase is known (Thomlinson et al., Biochemistry 1 (1962), 166-171). An alkaline phytase from lilly pollen has been described by Barrientos et al., Plant Physiol. 106 (1994), 1489-1495.

Amongst the bacteria, phytases have been described which are derived from Bacillus subtilis (Paver and Jagannathan, Journal of Bacteriology 151 (1982), 1102-1108) and Pseudomonas (Cosgrove, Australian Journal of Biological Sciences 23 (1970), 1207-1220).

There are several descriptions of phytase producing filamentous fungi. In particular, there are several references to phytase producing ascomycetes of the Aspergillus genus such as Aspergillus terreus (Yamada et al., Agric. Biol. Chem. 322 (1986), 1275-1282). Also, the cloning and expression of the phytase gene from Aspergillus niger var. awamori has been described (Piddington et al., Gene 133 (1993), 55-62). EP 0 420 358 describes the cloning and expression of a phytase of Aspergillus ficuum (niger). EP 0 684 313 describes the cloning and expression of phytases of the ascomycetes Myceliophthora thermophila and Aspergillus terreus.

EP 897 010 entitled “Modified phytases” discloses, i.a., certain variants of an Aspergillus fumigatus phytase. EP 897 985 entitled “Consensus phytases” discloses, i.a., a fungal consensus phytase which may be designed on the basis of, i.a., a multiple alignment of several ascomycete phytases. WO 99/48380 entitled “Thermostable phytases in feed preparation and plant expression” relates to certain aspects of using thermostable phytases. WO 00/143503 entitled “Improved phytases” relates i.a. to certain phytase variants of increased thermo-stability, which may be designed by a process similar to the one described in EP 897985.

A phytase derived from Peniophora lycii is disclosed in WO 98/28408, and certain variants thereof in WO 99/49022, as well as in WO 03/066847.

Phytase producing yeasts are also described: For example, EP 0 699 762 A2 describes the cloning and expression of a phytase of the yeast Schwanniomyces occidentalis.

For the use of phytase as a feed additive a thermostable product is needed which is not heat-inactivated during the required pelleting process at 80° C. to 90° C. As an indicator for the pelleting stability of the phytase, which is needed when using it as a feed additive, two parameters, the temperature optimum as well as the temperature stability are of high interest.

Thus, the technical problem underlying the present invention is the provision of a phytase with a high intrinsic thermostability.

This problem is solved by the provision of the embodiments as characterized in the claims.

Accordingly, the present invention relates to polynucleotides selected from the group consisting of
(a) polynucleotides comprising a nucleotide sequence encoding a polypeptide with the amino acid sequence of SEQ ID NO:2;
(b) polynucleotides comprising the nucleotide sequence of the coding region shown in SEQ ID NO:1;
(c) polynucleotides encoding a polypeptide, the amino acid sequence of which is at least 60% identical to the amino acid sequence shown in SEQ ID NO:2 and which has phytase activity;
(d) polynucleotides comprising a nucleotide sequence encoding a fragment of the polypeptide encoded by a polynucleotide of (a), (b) or (c) wherein said fragment has phytase activity;
(e) polynucleotides comprising a nucleotide sequence the complementary strand of which hybridizes to the polynucleotide of any one of (a), (b) and (d), wherein said nucleotide sequence encodes a protein having phytase activity; and
(f) polynucleotides comprising a nucleotide sequence that deviates from the nucleotide sequence defined in (e) by the degeneracy of the genetic code.

Consequently, the present invention relates to polynucleotides encoding a polypeptide having phytase activity, said polynucleotides preferably encoding a polypeptide comprising the amino acid sequence indicated in SEQ ID NO: 2. More preferably, the polynucleotide encodes residues 2 to 462 of the amino acid sequence shown in SEQ ID NO:2.

The phytase having the amino acid sequence as shown in SEQ ID NO:2 as well as a variant having amino acid residues 2 to 462 of SEQ ID NO:2 have been isolated from the Pichia guilliermondii strain LU124 (DSM 16949). In particular, the phytase which has been isolated from the cells by the purification method as described in the Examples has the amino acid sequence starting with residue 2 of SEQ ID NO:2, i.e. it lacks the N-terminal methionine, as N-terminal sequencing has revealed. The corresponding nucleotide sequence which has been isolated encodes a polypeptide having the amino acid sequence shown in SEQ ID NO:2. It has surprisingly been found that this phytase has a high intrinsic thermostability. Its temperature stability value (T 50), i.e. the temperature at which the enzyme still retains 50% of its activity, is about 74° C. The optimal reaction temperature is about 71° C. The pH optimum of the identified phytase is at about pH 4.0.

The identified phytase shows little homology to any of the known phytases, i.e. the highest homology found to a known phytase is about 50% on the amino acid level to the phytase from Schwanniomyces occidentalis (also known as Debaromyces castellii).

The present invention also relates to polynucleotides which encode a polypeptide, which has a homology, that is to say a sequence identity, of at least 60%, preferably of at least 70%, more preferably of at least 80%, even more preferably of at least 85% and particularly preferred of at least 90%, especially preferred of at least 95% and even more preferred of at least 98% to the entire amino acid sequence as indicated in SEQ ID NO: 2, the polypeptide having phytase activity.

Moreover, the present invention relates to polynucleotides which encode a polypeptide having phytase activity and the nucleotide sequence of which has a homology, that is to say a sequence identity, of at least 65%, preferably of at least 70%, more preferably of at least 80%, even more preferably of more than 85%, in particular of at least 90%, especially preferred of at least 95%, in particular of at least 97% and even more preferred of at least 98% when compared to the coding region of the sequence shown in SEQ ID NO:1.

Moreover, the present invention relates to polynucleotides which encode a polypeptide having phytase activity and the complementary strand of which hybridizes with a polynucleotide mentioned in any one of sections (a), (b) and (d), above.

The present invention also relates to polynucleotides, which encode a polypeptide having phytase activity and the sequence of which deviates from the nucleotide sequences of the above-described polynucleotides due to the degeneracy of the genetic code.

The invention also relates to polynucleotides comprising a nucleotide sequence which is complementary to the whole or a part of one of the above-mentioned sequences.

In the context of the present invention the term “hybridization” means hybridization under conventional hybridization conditions, preferably under stringent conditions, as for instance described in Sambrook and Russell (2001), Molecular Cloning: A Laboratory Manual, CSH Press, Cold Spring Harbor, N.Y., USA. In an especially preferred embodiment, the term “hybridization” means that hybridization occurs under the following conditions:

| | |
|---|---|
| Hybridization buffer: | 2 x SSC; 10 x Denhardt solution (Fikoll |
| | 400 + PEG + BSA; ratio 1:1:1); 0.1% SDS; |
| | 5 mM EDTA; 50 mM Na₂HPO₄; |
| | 250 μg/ml of herring sperm DNA; 50 μg/ml |
| | of tRNA; or |
| | 0.25 M of sodium phosphate buffer, |
| | pH 7.2; |
| | 1 mM EDTA |
| | 7% SDS |
| Hybridization temperature | 60° C. |
| T = | |
| Washing buffer: | 2 x SSC; 0.1% SDS |
| Washing temperature T = | 60° C. |

Polynucleotides which show the above indicated degree of homology or which hybridize with the polynucleotides of the invention can, in principle, encode a polypeptide having phytase activity from any organism expressing such polypeptides or can encode modified versions thereof.

Such polynucleotides can for instance be isolated from genomic libraries or cDNA libraries of organisms belonging to the prokaryotes or of organisms belonging to the eukaryotes, e.g. of bacteria, fungi, plants or animals. Preferably, such polynucleotides are of fungal origin, more preferred from a fungus belonging to the phylum of Ascomycota, even more preferred from a fungus belonging to the subphylum Saccharomyconita, particularly preferred of a fungus belonging to the class of Saccharomycetes. In a preferred embodiment the polynucleotides of the present invention can be isolated from a fungus of the order Saccharomycetales, even more preferred of the family Saccharomycetaceae, particularly preferred from a fungus of the genus Pichia, even more preferably from the species Pichia guilliermondii, most preferably from the strain Pichia guilliermondii LU124 (DSM16949). Alternatively, such polynucleotides can be prepared by genetic engineering or chemical synthesis.

Such polynucleotides may, e.g., be identified and isolated by using the polynucleotides described hereinabove or parts or reverse complements thereof, for instance by hybridization according to standard methods (see for instance Sambrook and Russell (2001), Molecular Cloning: A Laboratory Manual, CSH Press, Cold Spring Harbor, N.Y., USA). Polynucleotides comprising the same or substantially the same nucleotide sequence as indicated in SEQ ID NO: 1 or parts thereof can, for instance, be used as hybridization probes. The fragments used as hybridization probes can also be synthetic fragments which are prepared by usual synthesis techniques, and the sequence of which is substantially identical with that of a polynucleotide according to the invention.

The molecules hybridizing with the polynucleotides of the invention also comprise fragments, derivatives and allelic variants of the above-described polynucleotides encoding a polypeptide having phytase activity. Herein, fragments are understood to mean parts of the polynucleotides which are long enough to encode the described polypeptide, preferably showing the biological activity of a polypeptide of the invention as described above.

More preferably, such a fragment also has the characteristics with respect to T₅₀ value, temperature optimum and pH optimum as described herein further below. A particularly preferred fragment is a fragment comprising amino acid residues 2 to 462 of SEQ ID NO:2, i.e. a polypeptide which lacks the N-terminal methionine residue.

In this context, the term derivative means that the sequences of these molecules differ from the sequences of the above-described polynucleotides in one or more positions and show a high degree of homology to these sequences, preferably within the preferred ranges of homology mentioned above.

Preferably, the degree of homology is determined by comparing the respective sequence with the nucleotide sequence of the coding region of SEQ ID NO: 1 even more preferably with the coding region encoding amino acid residues 2 to 462 of the amino acid sequence shown in SEQ ID NO:1. In connection with amino acid sequences, the respective sequence is compared with the amino acid shown in SEQ ID NO:2, preferably with the residues 2 to 462 of SEQ ID NO:2. When the sequences which are compared do not have the same length, the degree of homology preferably refers to the percentage of nucleotide/amino acid residues in the shorter sequence which are identical to nucleotide/amino acid residues in the longer sequence. The degree of homology can be determined conventionally using known computer programs such as the DNASTAR program with the ClustalW analysis. This program can be obtained from DNASTAR, Inc., 1228 South Park Street, Madison, Wis. 53715 or from DNASTAR, Ltd., Abacus House, West Ealing, London W13 OAS UK (support@dnastar.com) and is accessible at the server of the EMBL outstation.

When using the Clustal analysis method to determine whether a particular sequence is, for instance, 80% identical to a reference sequence the settings are preferably as follows: Matrix: blosum 30; Open gap penalty: 10.0; Extend gap penalty: 0.05; Delay divergent: 40; Gap separation distance: 8 for comparisons of amino acid sequences. For nucleotide sequence comparisons, the Extend gap penalty is preferably set to 5.0.

Alternatively, and preferably, when determining the sequence identity of nucleotide or amino acid sequences, the GCG Wisconsin Package 10.3, Accelrys Inc., San Diego, Calif., is used. This package includes the GAP and BestFit programs.

When comparing amino acid sequences, it is preferable to use GAP, preferably with standard parameters; i.e. standard exchange matrix: Blosum 62; GAP-Weight: 8; GAP-Length:2. When comparing DNA sequences by using GAP and standard parameters, the standard exchange matrix is GCG nwsgapdna.cmp. The algorithm used in GAP is from Needleman and Wunsch (J. Mol. Biol. 48 (1970), 443-453).

Preferably, the degree of homology of the polynucleotide is calculated over the complete length of its coding sequence. It is furthermore preferred that such a polynucleotide, and in particular the coding sequence comprised therein, has a length of at least 300 nucleotides, preferably at least 500 nucleotides, more preferably of at least 750 nucleotides, even more preferably of at least 1000 nucleotides, particularly preferred of at least 1200 nucleotides and most preferably of at least 1300 nucleotides.

Preferably, sequences hybridizing to a polynucleotide according to the invention comprise a region of homology of at least 90%, preferably of at least 93%, more preferably of at least 95%, still more preferably of at least 98% and particularly preferred of at least 99% identity to an above-described polynucleotide, wherein this region of homology has a length of at least 1000 nucleotides, more preferably of at least 250 nucleotides, even more preferably of at least 500 nucleotides, particularly preferred of at least 100 nucleotides and most preferably of at least 1200 nucleotides.

Homology, moreover, means that there is a functional and/or structural equivalence between the corresponding polynucleotides or polypeptides encoded thereby. Polynucleotides which are homologous to the above-described molecules and represent derivatives of these molecules are normally variations of these molecules which represent modifications having the same biological function. They may be either naturally occurring variations, for instance sequences from other fungi, species, strains, etc., or mutations, and said mutations may have formed naturally or may have been produced by deliberate mutagenesis. Furthermore, the variations may be synthetically produced sequences. The allelic variants may be naturally occurring variants or synthetically produced variants or variants produced by recombinant DNA techniques. Deviations from the above-described polynucleotides may have been produced, e.g., by deletion, substitution, insertion and/or recombination.

The polypeptides encoded by the different variants of the polynucleotides of the invention possess certain characteristics they have in common. These include for instance biological activity, molecular weight, immunological reactivity, conformation, etc., and physical properties, such as for instance the migration behavior in gel electrophoreses, chromatographic behavior, sedimentation coefficients, solubility, spectroscopic properties, stability, pH optimum, temperature optimum etc.

In particular, a polypeptide encoded by a polynucleotide of the present application has phytase activity. In the context of the present application phytase activity means the capacity to effect the liberation of inorganic phosphate or phosphorous from various myo-inositol phosphates. Examples of such myo-inositol phosphates (phytase substrates) are phytic acid and any salt thereof, e.g. sodium phytate or potassium phytate or mixed salts. Also, any stereoisomer of the mono-, di-, tri-, tetra-, or penta-phosphates of myo-inositol might serve as a phytase substrate. A preferred phytase substrate is phytic acid or salts thereof.

The ENZYME site at the internet (http://www.expasy.ch/enzyme/) is a repository of information relative to the nomenclature of enzymes. It is primarily based on the recommendations of the Nomenclature Committee of the International Union of Biochemistry and Molecular Biology (IUB-MB) and it describes each type of characterized enzyme for which an EC (Enzyme Commission) number has been provided (Bairoch A., The ENZYME database, Nucl. Acids Res. 28 (2000), 304-305); see also the Handbook of Enzyme Nomenclature from NC-IUBMB (1992).

According, to the ENZYME site, two different types of phytases are known:
(i) a so-called 3-phytase (myo-inositol hexaphosphate 3-phosphohydrolase, EC 3.1.3.8); and
(ii) a so-called 6-phytase (myo-inositol hexaphosphate 6-phosphohydrolase, EC 3.1.3.26).

For the purpose of the present invention, both types are included within the meaning of the term “phytase”.

Phytase activity can be measured according to methods well known to the person skilled in the art. Preferably, phytase activity can be measured as described in the appended Examples (see Example 2a) or it can be determined according to “Determination of Phytase Activity in Feed by a Colorimetric Enzymatic Method”: Collaborative Interlaboratory Study Engelen et al.: Journal of AOAC International Vol. 84, No. 3, 2001.

One unit of phytase activity (=FTU) is defined as the amount of enzyme, which liberates 1 micromol of inorganic phosphorous per minute from 0.0051 mol/l of sodium phytate at pH 5.5 and 37° C.

The standard analytical method in this respect is based on the liberation of inorganic phosphate from sodium phytate added in excess. The incubation time at pH 5.5 and 37° C. is 60 min. The phosphate liberated is determined via a yellow molybdenium-vanadium complex and evaluated photometrically at a wavelength of 415 nm. A phytase standard of known activity is run in parallel for comparison. The measured increase in absorbance on the product sample is expressed as a ratio to the standard (relative method, the official AOAC method).

The phytase polypeptide encoded by a nucleic acid molecule according to the invention may be glycosylated or not glycosylated, preferably it is glycosylated.

Furthermore, the phytase polypeptide encoded by a nucleic acid molecule according to the present invention has a molecular weight when deduced from the amino acid sequence which is preferably between 50 and 53 kDa, more preferably between 51 and 52 kDa. The calculated molecular weight of the amino acid sequence shown in SEQ ID NO: 2 is 51986 Da. The protein having the amino acid sequence shown in SEQ ID NO: 2 but lacking the N-terminal methionine has a calculated molecular weight of 51855 Da.

More preferably, the molecular weight of the protein (in case it is glycosylated) when determined in SDS-PAGE is between 70 and 120 kDa, even more preferably between 80 and 110 kDa, and most preferably about 90 to 100 kDa.

The phytase proteins encoded by a polynucleotide of the present invention can preferably be isolated from crude cell extracts by the following chromatographic steps: (i) ion exchange chromatography on Q-Sepharose FF, (ii) size exclusion chromatography on a Superdex size exclusion chromatography column (Pharmacia) and (iii) high resolving ion exchange chromatography on a Mono Q column (Pharmacia), preferably as described in Example 1.

Moreover, the phytase protein encoded by a polynucleotide of the present invention has a temperature stability value (T50) of more than 65° C., preferably of more than 68° C., even more preferably of more than 70° C., particularly preferred of more than 72° C. and most preferably of about 74° C. The term “T50 value” means the temperature at which the residual activity, after preincubation at the indicated temperature, is 50%. The activity which refers to 100% activity is preferably determined at room temperature, most preferably after incubation for 20 minutes. The T50 value is preferably determined by using a crude cell extract of cells expressing the respective phytase protein, most preferably a crude cell extract prepared according to the method described in Example 1. The T50 value may also be determined using a purified enzyme preparation. In this case the T50 value may slightly differ from the T50 value determined by using a crude cell extract, i.e. it may be a little bit lower, probably due to the removal of stabilizing compounds, e.g. metal ions, during purification. The determination of the T50 value is most preferably carried out in acetate buffer at pH 5.5, especially preferred by using the conditions described in the Examples. The thermostability testing (i.e. determination of T50 value) comprises a stress test at different temperatures (for 20 minutes) and a subsequent measurement of the residual activity at standard conditions (37° C.).

Furthermore, a phytase protein encoded by a polynucleotide according to the present invention has an optimal reaction temperature which preferably lies in the range of 68° C. to 74° C., more preferably in the range of 69° C. to 73° C., even more preferably in the range of 70° C. to 72° C. and most preferably at about 71° C. The optimal reaction temperature is the temperature at which the phytase protein shows its highest activity. It is determined by measuring the phytase activity at different temperatures, preferably under the reaction conditions as described in the Examples. Most preferably, the optimal reaction temperature of the phytase is determined by using a crude cell extract of cells expressing the phytase. Particularly preferred the cell extract is prepared as described in Example 1. In particular, the measurement of the optimal reaction temperature and of the T50 value are preferably done with phytic acid as substrate via the so-called ascorbat (vitamin C) assay. This method quantifies the released phosphate from the substrate.

A phytase protein encoded by a polynucleotide of the present invention moreover shows preferably a pH optimum in the acidic range, more preferably in the range between pH 3.0 and 5.0, even more preferably in the range between pH 3.5 and 4.5 and most preferably a pH optimum of about pH 4.0. Moreover, the phytase protein shows significant activity in a range between about pH 2.6 to 6.0. The measurements for determining the phytase activity at different pH values are preferably carried out by using different buffer systems covering the pH range from pH 2.6 to 9.0. More preferably the measurements are carried out using the method as described in Example 2c).

The invention also relates to oligonucleotides specifically hybridizing to a polynucleotide of the invention. Such oligonucleotides have a length of preferably at least 10, in particular at least 15, and particularly preferably of at least 50 nucleotides. Advantageously, their length does not exceed a length of 1000, preferably 500, more preferably 200, still more preferably 100 and most preferably 50 nucleotides. They are characterized in that they specifically hybridize to the polynucleotides of the invention, that is to say that they do not or only to a very minor extent hybridize to nucleic acid sequences encoding another phytase. The oligonucleotides of the invention can be used for instance as primers for amplification techniques such as the PCR reaction or as a hybridization probe to isolate related genes. The hybridization conditions and homology values described above in connection with the polynucleotide encoding a polypeptide having phytase activity may likewise apply in connection with the oligonucleotides mentioned herein.

The polynucleotides of the invention can be DNA molecules, in particular genomic DNA or cDNA. Moreover, the polynucleotides of the invention may be RNA molecules. The polynucleotides of the invention can be obtained for instance from natural sources or may be produced synthetically or by recombinant techniques, such as PCR.

In another aspect, the present invention relates to recombinant nucleic acid molecules comprising the polynucleotide of the invention described above. The term “recombinant nucleic acid molecule” refers to a nucleic acid molecule which contains in addition to a polynucleotide of the invention as described above at least one further heterologous coding or non-coding nucleotide sequence. The term “heterologous” means that said polynucleotide originates from a different species or from the same species, however, from another location in the genome than said added nucleotide sequence. The term “recombinant” implies that nucleotide sequences are combined into one nucleic acid molecule by the aid of human intervention. The recombinant nucleic acid molecule of the invention can be used alone or as part of a vector.

For instance, the recombinant nucleic acid molecule may encode the polypeptide having phytase activity fused to a marker sequence, such as a peptide, which facilitates purification of the fused polypeptide. The marker sequence may for example be a hexa-histidine peptide, such as the tag provided in a pQE vector (Qiagen, Inc.) which provides for convenient purification of the fusion polypeptide. Another suitable marker sequence may be the HA tag which corresponds to an epitope derived from influenza hemagglutinin polypeptide (Wilson, Cell 37 (1984), 767). As a further example, the marker sequence may be glutathione-S-transferase (GST) which, apart from providing a purification tag, enhances polypeptide stability, for instance, in bacterial expression systems.

In a preferred embodiment, the recombinant nucleic acid molecules further comprise expression control sequences operably linked to the polynucleotide comprised by the recombinant nucleic acid molecule, more preferably these recombinant nucleic acid molecules are expression cassettes. The term “operatively linked”, as used throughout the present description, refers to a linkage between one or more expression control sequences and the coding region in the polynucleotide to be expressed in such a way that expression is achieved under conditions compatible with the expression control sequence.

Expression comprises transcription of the heterologous DNA sequence, preferably into a translatable mRNA. Regulatory elements ensuring expression in prokaryotic as well as in eukaryotic cells, preferably in fungal cells, are well known to those skilled in the art. They encompass promoters, enhancers, termination signals, targeting signals and the like. Examples are given further below in connection with explanations concerning vectors. In the case of eukaryotic cells, expression control sequences may comprise poly-A signals ensuring termination of transcription and stabilization of the transcript.

Moreover, the invention relates to vectors, in particular plasmids, cosmids, viruses, bacteriophages and other vectors commonly used in genetic engineering, which contain the above-described polynucleotides of the invention. In a preferred embodiment of the invention, the vectors of the invention are suitable for the transformation of fungal cells, cells of microorganisms, bacterial cells, animal cells or plant cells. In a particularly preferred embodiment such vectors are suitable for transformation of fungal cells, in particular yeast or filamentous fungi. Bacterial cells in this context are, e.g., bacteria of the genus Escherichia or Bacillus. Preferred are bacteria of the genus Bacillus because of their capability to secrete proteins into the culture medium. Other suitable bacteria are those from the genera Streptomyces and Pseudomonas.

Yeast cells in this context are, e.g., cells of the genera Saccharomyces, Kluyveromyces, Hansenula, Pichia, Yarrowia and Schizosaccharomyces. Preferred are yeast host cells of Saccharomyces cerevisiae, Kluyveromyces lactis (also known as Kluyveromyces marxianus var. lactis), Hansenula polymorpha, Pichia pastoris, Yarrowia lipolytica and Schizosaccharomyces pombe.

Filamentous fungal cells in this context are, e.g., cells of a genus selected from the group consisting of Aspergillus, Trichoderma, Fusarium, Disporotrichum, Penicillium, Acremonium, Neurospora, Thermoascus, Myceliophtora, Sporotrichum, Thielavia, and Talaromyces. More preferably the filamentous fungal cell is of the species Aspergillus oryzae, Aspergillus sojae, Aspergillus nidulans, or a species from the Aspergillus niger Group (as defined by Raper and Fennell, The Genus Aspergillus, The Williams & Wilkins Company, Baltimore, pp 293-344, 1965). These include but are not limited to Aspergillus niger, Aspergillus awamori, Aspergillus tubigensis, Aspergillus aculeatus, Aspergillus foetidus, Aspergillus nidulans, Aspergillus japonicus, Aspergillus oryzae and Aspergillus ficuum. In another preferred embodiment the filamentous fungal cell is a cell of the species Trichoderma reesei, Fusarium graminearum, Penicillium chrysogenum, Acremonium alabamense, Neurosporea crassa, Myceliophtora thermophilum, Sporotrichum cellulophilum, Disporotrichum dimorphosphorum or Thielavia terrestris.

In another preferred embodiment, the vectors further comprise expression control sequences operably linked to said polynucleotides contained in the vectors. These expression control sequence may be suited to ensure transcription and synthesis of a translatable RNA in prokaryotic or eukaryotic cells.

The expression of the polynucleotides of the invention in prokaryotic or eukaryotic cells, for instance in Escherichia coli, Saccharomyces cerevisiae or Pichia pastoris, is interesting because it permits a more precise characterization of the biological activities of the encoded polypeptide. In particular, recombinantly expressed polypeptide may be used to identify substrate compounds that are converted by its activity. Moreover, it is possible to express these polypeptides in such prokaryotic or eukaryotic cells which are free from interfering polypeptides. In addition, it is possible to insert different mutations into the polynucleotides by methods usual in molecular biology (see for instance Sambrook and Russell (2001), Molecular Cloning: A Laboratory Manual, CSH Press, Cold Spring Harbor, N.Y., USA), leading to the synthesis of polypeptides possibly having modified, preferably improved, biological properties, such as an increased specific activity, an increased T50 value or optimal reaction temperature. In this regard, it is on the one hand possible to produce deletion mutants in which polynucleotides are produced by progressive deletions from the 5′ or 3′ end of the coding DNA sequence, and said polynucleotides lead to the synthesis of correspondingly shortened polypeptides. On the other hand, the introduction of point mutations is also conceivable at positions at which a modification of the amino acid sequence for instance influences the biological activity, stability or the regulation of the polypeptide.

Mutants possessing a modified substrate or product specificity can be prepared. Furthermore, it is possible to prepare mutants having a modified activity-temperature-profile. Preferably, such mutants show an increased activity and a higher temperature stability (T50 value) and/or optimal reaction temperature.

Furthermore, in the case of expression, e.g., in fungal cells, in particular yeast cells, the introduction of mutations into the polynucleotides of the invention allows the gene expression rate and/or the activity of the polypeptides encoded by the polynucleotides of the invention to be reduced or increased.

For genetic engineering in prokaryotic cells, the polynucleotides of the invention or parts of these molecules can be introduced into plasmids which permit mutagenesis or sequence modification by recombination of DNA sequences. Standard methods (see Sambrook and Russell (2001), Molecular Cloning: A Laboratory Manual, CSH Press, Cold Spring Harbor, N.Y., USA) allow base exchanges to be performed or natural or synthetic sequences to be added. DNA fragments can be connected to each other by applying adapters and linkers to the fragments. Moreover, engineering measures which provide suitable restriction sites or remove surplus DNA or restriction sites can be used. In those cases, in which insertions, deletions or substitutions are possible, in vitro mutagenesis, “primer repair”, restriction or ligation can be used. In general, a sequence analysis, restriction analysis and other methods of biochemistry and molecular biology are carried out as analysis methods.

Additionally, the present invention relates to a method for producing genetically engineered host cells comprising introducing the above-described polynucleotides, recombinant nucleic acid molecules or vectors of the invention into a host cell.

Another embodiment of the invention relates to host cells, in particular prokaryotic or eukaryotic cells, genetically engineered with the above-described polynucleotides, recombinant nucleic acid molecules or vectors of the invention or obtainable by the above-mentioned method for producing genetically engineered host cells, and to cells derived from such transformed cells and containing a polynucleotide, recombinant nucleic acid molecule or vector of the invention. In a preferred embodiment the host cell is genetically modified in such a way that it contains a polynucleotide stably integrated into the genome. Preferably, the host cell of the invention is a bacterial, fungus, plant or animal cell. More preferred are filamentous fungal cells and most preferred are yeast cells. Bacterial cells in this context are, e.g., bacteria of the genus Escherichia or Bacillus. Preferred are bacteria of the genus Bacillus because of their capability to secrete proteins into the culture medium. Other suitable bacteria are those from the genera Streptomyces and Pseudomonas.

Yeast cells in this context are, e.g., cells of the genera Saccharomyces, Kluyveromyces, Hansenula, Pichia, Yarrowia and Schizosaccharomyces. Preferred are yeast host cells of Saccharomyces cerevisiae, Kluyveromyces lactis (also known as Kluyveromyces marxianus var. lactis), Hansenula polymorpha, Pichia pastoris, Yarrowia lipolytica and Schizosaccharomyces pombe.

Filamentous fungal cells in this context are, e.g., cells of a genus selected from the group consisting of Aspergillus, Trichoderma, Fusarium, Disporotrichum, Penicillium, Acremonium, Neurospora, Thermoascus, Myceliophtora, Sporotrichum, Thielavia, and Talaromyces. More preferably the filamentous fungal cell is of the species Aspergillus oryzae, Aspergillus sojae, Aspergillus nidulans, or a species from the Aspergillus niger Group (as defined by Raper and Fennell, The Genus Aspergillus, The Williams & Wilkins Company, Baltimore, pp 293-344, 1965). These include but are not limited to Aspergillus niger, Aspergillus awamori, Aspergillus tubigensis, Aspergillus aculeatus, Aspergillus foetidus, Aspergillus nidulans, Aspergillus japonicus, Aspergillus oryzae and Aspergillus ficuum. In another preferred embodiment the filamentous fungal cell is a cell of the species Trichoderma reesei, Fusarium graminearum, Penicillium chrysogenum, Acremonium alabamense, Neurosporea crassa, Myceliophtora thermophilum, Sporotrichum cellulophilum, Disporotrichum dimorphosphorum or Thielavia terrestris.

More preferably the polynucleotide can be expressed so as to lead to the production of a polypeptide having phytase activity. An overview of different expression systems is for instance contained in Methods in Enzymology 153 (1987), 385-516, in Bitter et al. (Methods in Enzymology 153 (1987), 516-544) and in Sawers et al. (Applied Microbiology and Biotechnology 46 (1996), 1-9), Billman-Jacobe (Current Opinion in Biotechnology 7 (1996), 5004), Hockney (Trends in Biotechnology 12 (1994), 456-463), Griffiths et al., (Methods in Molecular Biology 75 (1997), 427-440). An overview of yeast expression systems is for instance given by Hensing et al. (Antonie van Leuwenhoek 67 (1995), 261-279), Bussineau et al. (Developments in Biological Standardization 83 (1994), 13-19), Gellissen et al. (Antonie van Leuwenhoek 62 (1992), 79-93, Fleer (Current Opinion in Biotechnology 3 (1992), 486-496), Vedvick (Current Opinion in Biotechnology 2 (1991), 742-745) and Buckholz (Bio/Technology 9 (1991), 1067-1072). Also WO 99/32617 describes expression systems. The production of heterologous proteins in yeast or fungal cells is, e.g. described in Gellissen (Appl. Microbiol. Biotechnol. 54 (2000), 741-750), Gellissen et al. (Antonie van Leeuwenhoek 62 (1992), 79-93), Archer and Peberdy (Critical Reviews in Biotechnology 17 (1997), 273-306) and Radizio and Kuck (Process Biochem. 32 (1997), 529-539).

Expression vectors have been widely described in the literature. Generally, they contain not only a selection marker gene and a replication-origin ensuring replication in the host selected, but also a bacterial or viral promoter, and in most cases a termination signal for transcription. Between the promoter and the termination signal there is, in general, at least one restriction site or a polylinker which enables the insertion of a coding DNA sequence. The DNA sequence naturally controlling the transcription of the corresponding gene can be used as the promoter sequence, if it is active in the selected host organism. However, this sequence can also be exchanged for other promoter sequences. It is possible to use promoters ensuring constitutive expression of the gene and inducible promoters which permit a deliberate control of the expression of the gene. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of a downstream sequence are for instance the T7 promoter (Studier et al., Methods in Enzymology 185 (1990), 60-89), lacUV5, trp, trp-lacUV5 (DeBoer et al., in Rodriguez and Chamberlin (Eds), Promoters, Structure and Function; Praeger, New York, (1982), 462-481; DeBoer et al., Proc. Natl. Acad. Sci. USA (1983), 21-25), Ip1, rac (Boros et al., Gene 42 (1986), 97-100). Inducible promoters are preferably used for the synthesis of polypeptides. These promoters often lead to higher polypeptide yields than do constitutive promoters. In order to obtain an optimum amount of polypeptide, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (=isopropyl-β-D-thiogalactopyranoside) (deBoer et al., Proc. Natl. Acad. Sci. USA 80 (1983), 21-25). Termination signals for transcription are also described in the literature. Suitable promoters for the expression in Saccharomyces cerevisiae are, e.g. the ADH, GAL1, GAP, PHO5, ARG3, PGK or Mfalpha promoter. For methylotrophic yeast the following promoters are suitable: AOX1, AUG1 and GAP1 (for Pichia); MOX, FMD, GAP1, PMA1 and TRS1 (for Hansenula). Suitable promoters for expression in fungal cells are, e.g. the gpd promoter (from Aspergillus nidulans or Aspergillus niger or from the corresponding native gene of the host cell) and the glaA promoter, e.g. from A. niger. Other suitable promoters are, e.g., the cbh1 and the pki1 promoter for expression in Trichoderma reesei, the amy promoter for expression in Aspergillus oryzae or the alcA, suc1, aphA, tpiA or pkiA promoter for expression in A. niger.

The transformation of the host cell with a polynucleotide or vector according to the invention can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990 or in Guthrie and Fink: Guide to Yeast Genetics and Molecular Biology, Methods in Enzymology 169 (1991) Academic Press, San Diego, USA. A system for transformation and expression in Pichia pastoris is the expression kit K1710-01 (Invitrogen) which is commercially available. The transformation of A. niger is, e.g., described in Debets and Bos (FGN 33 (1986), 24) and in Werner et al. (Mol. Gen. Genet. 209 (1987), 71-77).The host cell is cultured in nutrient media meeting the requirements of the particular host cell used, in particular in respect of the pH value, temperature, salt concentration, aeration, antibiotics, vitamins, trace elements etc. The polypeptide according to the present invention can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Polypeptide refolding steps can be used, as necessary, in completing configuration of the polypeptide. Finally, high performance liquid chromatography (HPLC) can be employed for final purification steps.

Accordingly, the present invention also relates to a method for the production of a polypeptide encoded by a polynucleotide of the invention as described above in which the above-mentioned host cell is cultivated under conditions allowing for the expression of the polypeptide and in which the polypeptide is isolated from the cells and/or the culture medium. In a preferred embodiment, such a method allows the large scale production of the phytase according to the invention.

Moreover, the invention relates to a polypeptide which is encoded by a polynucleotide according to the invention or obtainable by the above-mentioned method for the production of a polypeptide encoded by a polynucleotide of the invention.

The polypeptide of the present invention may, e.g., be a naturally purified product or a product of chemical synthetic procedures or produced by recombinant techniques from a prokaryotic or eukaroytic host (for example, by bacterial, yeast, fungal, plant, insect and animal cells, in particular, mammalian cells in culture). Depending upon the host employed in a recombinant production procedure, the polypeptide of the present invention may be glycosylated or may be non-glycosylated. The polypeptide of the invention may include the initial methionine amino acid residue or may lack it. The polypeptide according to the invention may be further modified to contain additional chemical moieties not normally part of the polypeptide. Those derivatized moieties may, e.g., improve the stability, solubility, the biological half life or absorption of the polypeptide. The moieties may also reduce or eliminate any undesirable side effects of the polypeptide and the like. An overview for these moieties can be found, e.g., in Remington's Pharmaceutical Sciences (18^{th} ed., Mack Publishing Co., Easton, Pa. (1990)). Polyethylene glycol (PEG) is an example for such a chemical moiety which has been used for the preparation of therapeutic polypeptides. The attachment of PEG to polypeptides has been shown to protect them against proteolysis (Sada et al., J. Fermentation Bioengineering 71 (1991), 137-139). Various methods are available for the attachment of certain PEG moieties to polypeptides (for review see: Abuchowski et al., in “Enzymes as Drugs”; Holcerberg and Roberts, eds. (1981), 367-383). Generally, PEG molecules are connected to the polypeptide via a reactive group found on the polypeptide. Amino groups, e.g. on lysines or the amino terminus of the polypeptide are convenient for this attachment among others.

Furthermore, the present invention also relates to an antibody specifically recognizing a polypeptide according to the invention. The antibody can be monoclonal or polyclonal and can be prepared according to methods well known in the art. The term “antibody” also comprises fragments of an antibody which still retain the binding specificity.

The polypeptide according to the invention, its fragments or other derivatives thereof, or cells expressing them can be used as an immunogen to produce antibodies thereto. The present invention in particular also includes chimeric, single chain, and humanized antibodies, as well as Fab fragments, or the product of an Fab expression library. Various procedures known in the art may be used for the production of such antibodies and fragments.

Antibodies directed against a polypeptide according to the present invention can be obtained, e.g., by direct injection of the polypeptide into an animal or by administering the polypeptide to an animal, preferably a non-human animal. The antibody so obtained will then bind the polypeptide itself. In this manner, even a sequence encoding only a fragment of the polypeptide can be used to generate antibodies binding the whole native polypeptide. Such antibodies can then, e.g., be used to isolate the polypeptide from tissue expressing that polypeptide or to detect it in a probe. For the preparation of monoclonal antibodies, any technique which provides antibodies produced by continuous cell line cultures can be used. Examples for such techniques include the hybridoma technique (Köhler and Milstein, Nature 256 (1975), 495-497), the trioma technique, the human B-cell hybridoma technique (Kozbor et al., Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). Techniques describing the production of single chain antibodies (e.g., U.S. Pat. No. 4,946,778) can be adapted to produce single chain antibodies to immunogenic polypeptides according to the present invention. Furthermore, transgenic mice may be used to express humanized antibodies directed against immunogenic polypeptides of the present invention.

Furthermore, the invention relates to a method for producing a transformed host cell comprising the step of introducing at least one of the above-described polynucleotides, recombinant nucleic acid molecules or vectors of the invention into the host cell.

The present invention furthermore relates to a Pichia guilliermondii cell of the strain deposited under accession number DSM 16949 or a mutant or derivative thereof which has retained its capability of producing a phytase polypeptide according to the invention, preferably a phytase having a T50 value of about 74° C. and an optimal reaction temperature of about 71° C. when determined in a crude cell extract, more preferably a phytase having the characteristics of the phytase the purification and biochemical characterization of which is described in the Examples.

Moreover, the present invention also relates to a method for preparing a phytase comprising the steps of cultivating a Pichia guilliermondii cell as described above under conditions allowing expression of the phytase and recovering the phytase from the culture, in particular from the cells. In a preferred embodiment such a process also comprises the step(s) of further purifying the phytase, e.g., as described in the Examples. In a further preferred embodiment, the process allows the large scale production of the phytase protein. The phytase obtainable, obtained or produced by this method is also an object of the present invention.

The present invention furthermore relates to a composition comprising at least one polypeptide according to the present invention. Preferably such a composition is a food or feed or an additive for food or feed. A “feed” and a “food”, respectively, means any natural or artificial diet, meal or the like or components of such meals intended or suitable for being eaten, taken in, digested, by an animal and a human being, respectively.

The phytase according to the invention may exert its effect in vitro or in vivo, i.e. before intake or in the stomach of the individual, respectively. A combined action is also possible.

A composition according to the invention comprising a phytase may, e.g., be liquid or dry.

A liquid composition may only contain the phytase enzyme, preferably in a highly purified form. Usually, however, a stabilizer such as glycerol, sorbitol or mono propylene glycol is added. The liquid composition may also comprise other additives, such as salts, sugars, preservatives, pH-adjusting agents, proteins or a phytase substrate. Typical liquid compositions are aqueous or oil-based slurries. The liquid compositions can be added to a food or feed after an optional pelleting thereof.

Dry compositions may be spray-dried compositions. In this case the composition need not contain anything more than the enzyme in a dry form. Usually, however, dry compositions are so-called granulates which may readily be mixed with e.g. food or feed components, or more preferably, form a component of a premix. The particle size of the enzyme granulates preferably is compatible with that of the other components of the mixture. This provides a safe and convenient means of incorporating enzymes into e.g. animal feed.

The preparation of granulates is known to the person skilled in the art.

Agglomeration granulates are, e.g., generally prepared by using agglomeration technique in a high shear mixer (e.g. Lödige) during which a filler material and the enzyme are co-agglomerated to form granules. Absorption granulates are generally prepared by having cores of a carrier material to absorb the enzyme and/or be coated with the enzyme.

Examples for filler materials are salts such as disodium sulphate. Other fillers are kaolin, talc, magnesium aluminium silicate and cellulose fibres. Optionally, also binders such as dextrins are included in the agglomeration granulates.

Examples for carrier materials are starch, e.g. from cassaya, corn, potato, rice and wheat or salts.

The granulates may be coated with a coating mixture. Such a mixture comprises coating agents, preferably hydrophobic coating agents, such as hydrogenated palm oil and beef tallow, and if desired other additives, such as calcium carbonate or kaolin.

A phytase composition according to the invention may furthermore contain other substituents such as colouring agents, aroma compounds, stabilizers, minerals, vitamins, other feed or food enhancing enzymes, i.e. enzymes that enhances the nutritional properties of feed/food, etc.

The term “food or feed additive” means an essentially pure compound or a multi component composition intended for or suitable for being added to food or feed. In particular, it is a substance which by its intended use is becoming a component of a food or feed product or affects any characteristics of a food or feed product. It is preferably composed as indicated for a phytase composition above. A typical additive usually comprises one or more compounds such as vitamins, minerals or feed enhancing enzymes and suitable carriers and/or excipients.

In a preferred embodiment, the phytase composition of the present invention additionally comprises an effective amount of one or more feed enhancing enzymes. Such enzymes are known in the art and include, e.g., α-galactosidases, β-galactosidases, in particular lactases, other phytases, β-glucanases, in particular endo-β-1,4-glucanases and endo-β-1,3(4)-glucanases, cellulases, xylosidases, galactanases, in particular arabinogalactan endo-1,4-β-galactosidases and arabinogalactan endo-1,3-β-galactosidases, endoglucanases, in particular endo-1,2-β-glucanase, endo-1,3-α-glucanase, and endo-1,2-β-glucanase, pectin degrading enzymes, in particular pectinases, pectinesterases, pectin lyases, polygalacturonases, arabinanases, rhamnogalacturonases, rhamnogalacturonan acetyl esterases, rhamnogalacturonan-α-rhamnosidase, pectate lyases, and α-galacturonisidases, mannanases, β-mannosidases, mannan acetyl esterases, xylan acetyl esterases, proteases, xylanases, arabinoxylanases and lipolytic enzymes such as lipases, phospholipases and cutinases.

An animal feed additive according to the invention can be supplemented to the animal, in particular the mono-gastric animal, before or simultaneously with the diet. Preferably, it is supplemented to the animal simultaneously with the diet. More preferably, it is added to the diet in the form of a granulate or a stabilized liquid.

A composition according to the invention comprises an effective amount of the phytase. An effective amount in food or feed is preferably from about 10-20.000, preferably from about 10 to 10.000, in particular from about 100 to 5.000, especially from about 100 to about 2.000 FTU/kg feed or food.

A composition according to the invention can comprise a phytase according to the invention in any possible form. The phytase may be, e.g., in the form of cell extracts, culture supernatants, culture broth comprising cells expressing the phytase, cells expressing the phytase or biomass derived from such cells. Preferably the phytase is purified, e.g., partially purified. Most preferably, it is highly purified. “Highly purified” in this context means at least 80% pure, preferably at least 90% pure, more preferably at least 95% pure and most preferably at least 99% pure.

The present invention also relates to a process for preparing a feed or food comprising the steps of adding a polypeptide according to the invention to the feed or food components. The adding can be carried out according to methods well known to the person skilled in the art.

Finally, the present invention also relates to the use of a polypeptide of the present invention for liberating inorganic phosphate from phytic acid or phytate as well as to the use of such a polypeptide in the preparation of a food or feed or as an additive for a food or feed.

The strain Pichia guilliermondii LU124 was deposited in accordance with the requirements of the Budapest Treaty at the Deutsche Sammlung für Mikroorganismen und Zellkulturen (DSMZ) in Braunschweig, Federal Republic of Germany on Nov. 29, 2004 under accession number DSM 16949.

The Figures show:

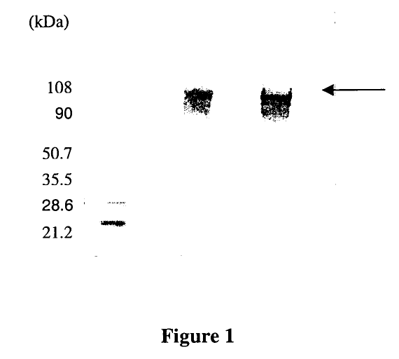

FIG. 1 shows the SDS-PAGE analysis of purified phytase from the Pichia guilliermondii strain LU124 (DSM 16949). The arrow indicates the phytase bands.

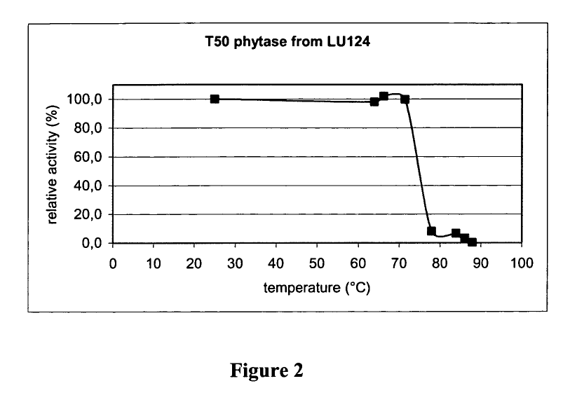

FIG. 2 shows the results of the determination of the thermostability of phytase from the Pichia guilliermondii strain LU124 (DSM 16949) before purification. The measurements were carried out in acetate buffer at pH 5.5. The T50 value in crude extract was 74° C.

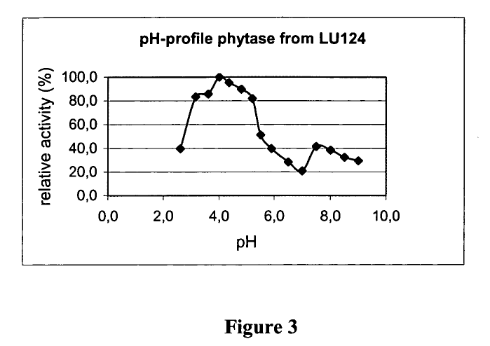

FIG. 3 shows the pH profile of phytase from Pichia guilliermondii LU124 (DSM 16949).

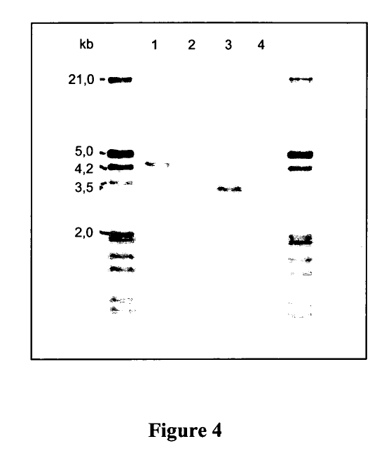

FIG. 4 shows the Southern blot analysis of Pichia guilliermondii chromosomal DNA digested with EcoRI (lane 1) and HindIII (lane 3) and probed with the amplified phytase PCR product.

The following Examples serve to further illustrate the invention.

### EXAMPLE 1

### Protein Isolation

Unless otherwise specified, all chemicals are used in the highest purity available and obtained from Sigma. All medium components are sterilized, either by heat (20 minutes at 1.5 bar and 121° C.) or by sterile filtration. The components can either be sterilized together or, if necessary, separately.

Yeast strain Pichia guilliermondii LU124 (DSM 16949) was cultivated in a phosphate depleted Yeast-Peptone (YP) medium in an ISF 100-Fermenter (Infors) at 3-L scale under the following conditions:

### Yeast-Peptone (YP) Medium, Phosphate Depleted:

1% Yeast extract (Difco), 1% Bacto peptone (Difco), 2% Glucose, 2 g/l Phytic acid, pH 5.5
the 10× stock solutions for yeast extract and Bacto peptone were phosphate depleted by adding 10 mM MgCl₂, adjusting pH to 8.6, stirring over night at 4° C. and clearing of the solution by centrifugation (10 min, 4000 rpm)

### Fermentation Parameters:

Start volume: 3.5 L medium
Aeration rate: 2 L/min (const.)
Rotational frequency: 300 rpm (start)
PO₂ regulation via rotational frequency
PO₂ minimum: 30%
Temperature: 30° C.
pH 6, regulated with 10% HCl/25% NaOH
Feed (50% glucose) as required

After 48 h the biomass was harvested by centrifugation (10000 g, 4° C.): 360 g wet cells of LU124 were suspended in 1 l of 20 mM sodium acetate at pH 5.0. The volume was homogenized two times at 1500 bar in a microfluidizer (Z04). Protein precipitation is observed. The cell debris was removed by centrifugation. A final protein concentration of 0.7 mg/ml was reached (conductance 3 mS).

This intracellular protein was purified to near homogeneity by several chromatographic steps including ion exchange chromatography (Q-Sepharose FF column), size exclusion chromatography (Superdex size exclusion chromatography column, Pharmacia), and high resolving ion exchange chromatography (MonoQ column, Pharmacia):

### a) Ion Exchange Chromatography

A column of Q-Sepharose FF with a volume of 400 ml (5 cm in diameter) was used. The column was equilibrated with buffer A (20 mM sodium acetate, pH 4.9). The homogenate (1340 ml) was applied at a flow rate of 10 ml/min. After washing with 2 column volumes buffer A a linear gradient to 100% buffer B (buffer A with 1.5M NaCl) in the course of 120 minutes was applied. Active fractions (150 ml, Fr. 45-60) were collected (53 mg protein).

### b) Size Exclusion Chromatography

The active phytase was concentrated on amicon ultrafiltration (YM10) membranes to a volume of 5 to 10 ml. This volume was after centrifugation applied to a Superdex size exclusion chromatography column (Pharmacia) at a flow rate of 1 ml/min in buffer A. Active fractions were collected (9.7 mg protein, 55 ml).

### c) High Resolving Ion Exchange Chromatography

A Mono-Q (1 ml, Pharmacia) column was equilibrated with buffer A. At two separate runs 25 ml of the active fractions from the previous column were applied at 1 ml/min. After washing for ten minutes with buffer A the protein was eluted with a linear gradient to 100% buffer B in 60 minutes. Active fractions (7.8) were collected.

The isolated protein showed a size of about 100 kDa in SDS-Page analysis (see FIG. 1)

### EXAMPLE 2

### Characterization of the Isolated Enzyme

### 2a) Enzymatic Activity

Enzymatic activity was measured with phytic acid as substrate and at an appropriate level of phytase activity (standard: 0.6 U/ml) in a 250 mM acetic acid/sodium acetate/Tween 20 (0.1%), pH 5.5 buffer. The assay was standardized for application in micro titer plates (MTP).
10 μl of the enzyme solution was mixed with 140 μl 6.49 mM phytate solution in 250 mM sodium acetate buffer, pH 5.5 (phytate: dodecasodium salt of phytic acid). After incubation for 1 hour at 37° C., the reaction was stopped by addition of an equal volume of 15% trichloroacetic acid (150 μl). An aliquot of this mixture (20 μl) was transferred to 280 μl solution containing 0.32 N H₂SO₄, 0.27% ammonium molybdate and 1.08% ascorbic acid, followed by incubation for 25 minutes at 50° C. The absorbance of the blue colored solution was measured at 820 nm.

### 2b) Determination of the Temperature Profile

The temperature optimum was determined by measuring the phytase activity at different temperatures. The thermostability testing comprises a stress test at different temperatures (for 20 min) and a subsequent measurement of the residual activity at standard conditions (37° C.) as described under 2a). The T50 value describes the temperature at which the residual activity is 50%.
The phytase from P. guilliermondii has an optimal reaction temperature of 71° C. in the crude extract and a T50 value of 74° C. (see FIG. 2). These values are about 15° C. higher compared to an Aspergillus ficuum phytase, which is commercially available as NATUPHOS™ (BASF AG).

### 2c) Determination of DH-Profile

In the assay for the different pH value 4 different buffer systems are used:

| | |
|---|---|
| Glycine buffer (250 mM): | pH 2.6-3.2 |
| Acetate buffer (250 mM): | pH 3.6-5.5 |
| Imidazole buffer (250 mM): | pH 5.9-7.0 |
| Tris buffer (250 mM): | pH 7.5-9.0 |

50 μl enzyme solution were added to 700 μl 6.49 mM sodium phytate in buffer with the desired pH value and incubated for 1 hour at 37° C. The enzyme activity was then measured as described under 2a). The results are shown in FIG. 3.

### EXAMPLE 3

### Determination of the N-Terminal Sequence and Tryptic Fragments

The protein bands at 90 to 100 kDa from the SDS page gel (see FIG. 1) were cut out, washed, eluted and digested with trypsin. The peptides were separated on a reversed phase capillary HPLC (UltiMate, Dionex; C18) collected and the sequences were determined by automated Edman degradation (cLC-494, Applied Biosystems). The N-terminal sequence VAIQKALVPGLYLASNY-RDVATPELAARDQYNIV was determined from a blot.

### EXAMPLE 4

### Cloning and Sequencing of Phytase from P. quilliermondii

Unless otherwise specified, all DNA manipulations and transformations were performed using standard methods of molecular biology (Sambrook et al. (1989) Molecular Cloning: A laboratory manual. Cold Spring Harbor lab. Cold Spring Harbor N.Y.; Ausubel, F. M. et al. (eds.) “Current protocols in Molecular Biology”, John Wiley and Sons, 1995; Innis et al. (1990) PCR Protocols. A Guide to Methods and Applications, Academic Press).

### 4a) Construction of Degenerate Oligonucleotides

The first degenerated oligo Haf236: 5′-GTNGCNATHCARAARGC-3′ (SEQ ID NO:3) was deduced from the N-terminal sequence: VAIQKA. The amino acid sequence QNEENY, obtained from an sequenced tryptic fragment of the purified enzyme was used for the creation of the reverse oligo Haf259 5′-RTARTTYTCYTCRTTYTG-3′ (SEQ ID NO:4).

### 4b) Cloning

Preparation of Chromosomal DNA
Cells were cultivated overnight in 20 ml YPD medium (1% Yeast extract, 1% Bacto peptone, 2% Glucose) at 30° C. and harvested by centrifugation. 200 mg of the pellet were resuspended in 800 μl H₂O. A red Ribolyser tube (Hybaid, matrix C) was filled with 700 μl cell suspension and 780 μl phenol/chloroform (TE buffered, pH 7.5). The cells were disrupted at level 6 for 2×30 s (in-between cooled on ice) and centrifuged (5 min, 10000 rpm, 4° C.). 650 μl supernatant was digested with 2 μl RNAse (10 mg/ml) for 30 min at 37° C. and afterwards precipitated with 65 μl 3 M Na-acetate and 1.3 ml ethanol. The pelletized DNA (10 min, 13000 rpm, 4° C., Biofuge Fresco, Heraeus, Hanau, Germany) was washed with 70% ethanol, air dried and re-suspended in 100 μl H₂O (=chromosomal DNA).

PCR
1 μl template (=chromosomal DNA, isolated as described above), 2 μl of each oligo Haf236/Haf259 and Haf236/Haf257), 0.5 μl dNTPs (10 mM), 5 μl buffer, 1 μl Pfu ultra polymerase (Stratagene), and water added to a final volume of 50 μl were mixed. PCR program parameters: 94° C., 5 min; (94° C., 30 s; 45° C., 30 s; 72° C., 90 s)×30 cycles; 72° C. 10 min. Due to the used low annealing temperature of 45° C., several bands were detected on the electrophoresis gel. All PCR fragments were isolated from the gel (QiaEXII Gel Extraction Kit, Quiagen and ligated into the EcoRV restriction site of a pBlueScript vector (Stratagene) using standard methods.
The inserts of the obtained plasmids were sequenced according to Sanger et al., Proceedings of the National Academy of Sciences USA 74 (1997), 5463-5467. An ABI Prism 377 (PE Applied Biosystems, Weiterstadt) was used for the separation and analysis of the sequencing reaction.
The translation of one of the obtained DNA sequences resulted in a continuous peptide which contains the previously determined amino acid sequence of the N-terminus.

Amplification of Full Length Sequence with Inverse PCR
First of all, a Southern blot analysis (Sambrook et al. (1989), Molecular Cloning. A Laboratory Manual, Cold Spring Harbor) was performed, using the amplified fragment as a probe. Chromosomal DNA was digested with several restriction enzymes (Roche Diagnostics, Mannheim). The digestion with HindIII and EcoRI resulted in clear hybridization bands with a moderate fragment size of 3.4 kb and 4.4 kb respectively (FIG. 4). Since HindIII cuts the Pichia phytase within the sequenced area at the 3′-end, the fragment includes approx. 2.5 kb upstream of the sequenced 5′-end. The EcoRI fragment included sequences from both sides of the known fragment.
The HindIII digestion and the EcoRI digestion were used for the inverse PCR. Therefore DNA of the appropriate size (around 3.4 kb and 4.4 kb) was isolated from a gel and circularized in a ligation reaction. The PCR was performed with Haf271 5′-GTGGTTGTACTTTGCTCTG-3′ and Haf272 5′-CCCGATTATCTGGACGAG-3′ which were complementary to the initial sequenced PCR fragment and extending outwards.

Inverse PCR
The chromosomal DNA (3 μl) was digested with 3 μl enzyme in a total volume of 50 μl for 3 h at the recommended temperature. After gel electrophoresis, DNA fragments with approximately the size, which was determined by a Southern analysis, were isolated with a GFX-column (GFX DNA and Gel Band Purification Kit, Amersham Bioscience, UK). 30 μl purified DNA were ligated with 2 μl ligase (Rapid ligation kit, Roche Diagnostics, Mannheim) in a total volume of 40 μl for 15 min and afterwards purified with a GFX column. This DNA (10 μl) was used as template in a standard 50 μl PCR (Innis et al. (1990) PCR Protocols. A Guide to Methods and Applications, Academic Press). PCR program: 94° C., 5 min; (94° C., 30 s; 58° C., 30 s; 72° C., 270 s)×25 cycles; 72° C. 10 min
The reactions for both digestions (HindIII, EcoRI) resulted in a PCR product with the expected length, which was cloned in a pBlueScript vector and sequenced.
As expected, the 5′-region of the phytase was gained from the HindIII fragment. The EcoRI fragment delivered the missing 3′-end of the phytase. The complete sequence of the phytase from Pichia guilliermondii was assembled from the different PCR products and is shown in SEQ ID NO:1. The encoded amino acid sequence is shown in SEQ ID NO:2.

1. A polynucleotide selected from the group consisting of
(a) polynucleotides comprising a nucleotide sequence encoding a polypeptide with the amino acid sequence of SEQ ID NO:2;
(b) polynucleotides comprising the nucleotide sequence of the coding region shown in SEQ ID NO: 1;
(c) polynucleotides encoding a polypeptide the amino acid sequence of which is at least 60% identical to the amino acid sequence shown in SEQ ID NO: 2 and which has phytase activity;
(d) polynucleotides comprising a nucleotide sequence encoding a fragment of the polypeptide encoded by a polynucleotide of (a), (b) or (c) wherein said fragment has phytase activity;
(e) polynucleotides comprising a nucleotide sequence the complementary strand of which hybridizes to the polynucleotide of any one of (a), (b) and (d), wherein said nucleotide sequence encodes a protein having phytase activity; and
(f) polynucleotides comprising a nucleotide sequence that deviates from the nucleotide sequence defined in (e) by the degeneracy of the genetic code.

2. The polynucleotide of claim 1 which is DNA or RNA.

3. A recombinant nucleic acid molecule comprising the polynucleotide of claim 1.

4. The recombinant nucleic acid molecule of claim 3 further comprising expression control sequences operably linked to said polynucleotide.

5. A vector comprising a polynucleotide selected from the group consisting of the polynucleotide of claim 1, the polynucleotide of claim 2, the recombinant nucleic acid molecule of claim 3 and the recombinant nucleic acid molecule of claim 4.

6. The vector of claim 5 further comprising expression control sequences operably linked to said polynucleotide.

7. A method for producing genetically engineered host cells comprising introducing into a host cell a polynucleotide selected from the group consisting of the polynucleotide of claim 1, the polynucleotide of claim 2, the recombinant nucleic acid molecule of claim 3, the recombinant nucleic acid molecule of claim 3, the vector of claim 5, and the vector of claim 6.

8. A host cell which is genetically engineered with a polynucleotide selected from the group consisting of the polynucleotide of claim 1, the polynucleotide of claim 2, the recombinant nucleic acid molecule of claim 3, the recombinant nucleic acid molecule of claim 3, the vector of claim 5, and the vector of claim 6.

9. The host cell of claim 8 which is a bacterial, yeast, fungus, plant or animal cell.

10. A method for the production of a polypeptide encoded by the polynucleotide of claim 1 in which the host cell of claim 9 is cultivated under conditions allowing for the expression of the polypeptide and in which the polypeptide is isolated from at least one of the cells and the culture medium.

11. A polypeptide encoded by the polynucleotide of claim 1.

12. A Pichia guilliermondii cell of the strain deposited under accession number DSM 16949 or a mutant or derivative thereof which has retained the capability of producing a phytase having a T50 value of about 74° C. and an optimal reaction temperature of about 71° C. when determined in a crude cell extract.

13. A phytase obtained from a Pichia cell according to claim 12.

14. An antibody specifically recognizing the polypeptide of claim 11.

15. A composition comprising a polypeptide selected from the group consisting of the polypeptide of claim 11 and the phytase of claim 13.

16. The composition of claim 15 which is a feed, food or an additive for a feed or a food.

17. A process for preparing a feed or a food comprising the step of adding the polypeptide of claim 11 or the phytase of claim 13 to the feed or food components.

18. (canceled)

19. A polypeptide obtained by the method of claim 10.

20. A method for liberating inorganic phosphate from phytic acid comprising the use of the polypeptide of claim 11 or the phytase of claim 13.

21. A host cell that is genetically engineered with a polynucleotide obtained by the method of claim 7.
